(19) United States
(12) Patent Application Publication
(10) Pub. No.: US 2025/0312192 A1
(43) Pub. Date: Oct. 9, 2025

(54) Methods and Apparatus to Remove Corneal Endothelium and Inner Corneal Lesions with a Cannula

(71) Applicant: Martin de la Presa Pothier, Benton, KS (US)

(72) Inventor: Martin de la Presa Pothier, Benton, KS (US)

(21) Appl. No.: 19/016,988

(22) Filed: Jan. 10, 2025

Related U.S. Application Data
(60) Provisional application No. 63/575,683, filed on Apr. 6, 2024.
(60) Provisional application No. 63/707,596, filed on Oct. 15, 2024.

(51) Int. Cl. A61F 9/007; A61B 17/34

(52) U.S. Cl. CPC A61F 9/00736; A61B 17/3421; A61B 2017/3454; A61B 2217/007

(57) ABSTRACT

A medical device assembly comprising, a connector including a body, a proximal end, and a distal end, a cannula including a proximal section, a medial section, and a distal section, wherein the proximal section is secured to the connector body, wherein the medial section extends at an angle from the proximal section, wherein the distal section extends from the medial section and has an arcuate shape following the curvature of the inner cornea, and wherein the canula has at least one internal lumen.

### RELATED CASES

This application claims priority from U.S. Provisional Application No. 63/575,683 filed on Apr. 6, 2024, and U.S. Provisional Application No. 63/707,596 filed on Oct. 15, 2024. The disclosure of that application is expressly incorporated herein by reference.

### FIELD OF THE INVENTION

The present invention relates to ophthalmic surgical instruments and, more specifically, to a novel cannula designed for the precise removal of corneal endothelium and inner corneal lesions while preserving Descemet's membrane.

### BACKGROUND OF THE INVENTION

Corneal transplantation, also known as keratoplasty, is a well-established surgical procedure for treating corneal diseases and injuries. In traditional full-thickness corneal transplants, the entire cornea, including the endothelial layer, is replaced. However, advancements in surgical techniques have led to the development of selective endothelial replacement procedures, which aim to specifically target and replace only the damaged endothelial and/or inner corneal layer while leaving the rest of the cornea intact.

Various surgical instruments and techniques have been proposed for the selective removal of corneal endothelium and inner corneal lesions. Manual techniques involving the use of forceps or blades are limited by their inability to precisely target and remove the endothelium and inner corneal lesions without damaging the underlying Descemet's membrane. Automated devices have also been utilized for endothelial removal but suffer from drawbacks such as high cost, complexity, and risk of tissue damage.

There remains a need for a surgical instrument capable of selectively removing corneal endothelium and inner corneal lesions with precision while minimizing damage to Descemet's membrane. Such an instrument should be easy to use, cost-effective, and compatible with existing surgical procedures for corneal transplantation and endothelial therapy.

### SUMMARY OF THE INVENTION

A medical device assembly comprising, a connector including a body, a proximal end, and a distal end, a cannula including a proximal section, a medial section, and a distal section, wherein the proximal section is secured to the connector body, wherein the medial section extends at an angle from the proximal section, wherein the distal section extends from the medial section and has an arcuate shape following the curvature of the inner cornea, and wherein the canula has at least one internal lumen.

### BRIEF DESCRIPTION OF THE DRAWINGS

FIG. 1A is a perspective view of one embodiment of the present invention.

FIG. 1B is a perspective view of one embodiment of the present invention.

FIG. 2 is a perspective view of one embodiment of the present invention.

FIG. 3A is a side view of one embodiment of the present invention.

FIG. 3B is a top view of one embodiment of the present invention.

FIG. 4A is a side view of one embodiment of the present invention.

FIG. 4B is a top view of one embodiment of the present invention.

FIG. 5A is a perspective view of one embodiment of the present invention.

FIG. 5B is a perspective view of one embodiment of the present invention.

FIG. 6A is a cross-section view of one embodiment of the present invention inserted into an eye.

FIG. 6B is a cross-section view of one embodiment of the present invention inserted into an eye.

FIG. 6C is a cross-section illustration of a human eye.

FIG. 6D is an exploded view of a cross-section of a human cornea.

FIG. 7A is a front view of one embodiment of the present invention inserted into an eye.

FIG. 7B is a front view of one embodiment of the present invention inserted into an eye.

FIG. 7C is a front view of one embodiment of the present invention inserted into an eye.

FIG. 7D is a front view of one embodiment of the present invention inserted into an eye.

FIG. 8A is a perspective view of one embodiment of the present invention illustrating an angle of use.

FIG. 8B is a perspective view of one embodiment of the present invention illustrating an angle of use.

FIG. 8C is a perspective view of one embodiment of the present invention illustrating an angle of use.

FIG. 9A is a perspective view of one embodiment of the present invention inserted into an eye illustrating an angle of use.

FIG. 9B is a perspective view of one embodiment of the present invention inserted into an eye illustrating an angle of use.

FIG. 9C is a perspective view of one embodiment of the present invention inserted into an eye illustrating an angle of use.

FIG. 9D is a perspective view of one embodiment of the present invention inserted into an eye illustrating an angle of use.

### DETAILED DESCRIPTION OF THE INVENTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope of the present disclosure. In addition, descriptions of well-known functions and constructions may be omitted for clarity and conciseness.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The terms and words used in the following description and claims are not limited to the bibliographical meanings, but are merely used by the inventor to enable a clear and consistent understanding of the present disclosure. Accordingly, it should be apparent to those skilled in the art that the following description of various embodiments of the present disclosure is provided for illustration purposes only and not for the purpose of limiting the present disclosure as defined by the appended claims and their equivalents. Depending on the context of the sentence, the reading of this patent specification should be done with the same concept as “reject the absurd”, wherein if possible, a specification should be construed so as not to lead to a foolish result or one which the patentee could not have contemplated.

It is to be understood that the singular forms “a”, “an,” and “the” include plural referents unless the context clearly dictates otherwise. Thus, for example, reference to “a spring” includes reference to one or more of such springs.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention offers significant advancement in the field of ophthalmic surgery by providing a versatile and efficient tool for selective corneal endothelial removal, with potential applications in corneal transplantation and endothelial cellular therapy. The present invention addresses previous challenges by providing a novel medical device assembly specifically designed for the precise removal of corneal endothelium and inner corneal lesions while preserving Descemet's membrane (even while engaging with, polishing, and even shaving down the membrane). The medical device assembly can include a lure lock hub for connection to irrigation tubing, a cannula extending from the lure lock hub, the cannula including a proximal section extending into the anterior chamber of an eye, a medial section which elevates a distal section to the inner layer of the cornea, the distal section following the curvature of the inner cornea to remove endothelium and inner corneal lesions with gentle wiping motions initiated with the rotation of the device.

The unique design of the medical device assembly allows for controlled and targeted removal of corneal endothelium and inner corneal lesions and Descemet membrane lesions, with or without the presence of a sleeve, ensures smooth and gentle tissue interaction. Additionally, the ability to connect the connector to constant irrigation helps maintain the depth of the anterior chamber during the procedure, further enhancing surgical precision and safety.

The present invention involves a medical device assembly 10 comprising a connector 20 including a body 21, a proximal end 22, and a distal end 23, a cannula 30 including a proximal section 40, a medial section 50, and a distal section 60, wherein the proximal section 40 is secured to the connector body 21, wherein the medial section 50 extends at an angle from the proximal section 40, wherein the distal section 60 extends from the medial section 50 and has an arcuate shape following the curvature of the inner cornea, and wherein the canula 30 has at least one internal lumen 37. Looking now to the figures, the connector 20 is illustrated having a body 21, a proximal end 22 and a distal end 23. In some embodiments the connector 20 may also either be a handle, a lure lock hub, or a combination of both. In some embodiments, the connector 20, as a lure lock hub, may be designed with a finger grip to prevent rotation during use of the medical device assembly 10. In some embodiments, the connector 20 is configured to connect to irrigation (constant or intermittent as needed) to maintain a depth of the anterior chamber. In some embodiments, the connector 20, as a lure lock hub, may be capped to prevent the egress of fluid from the anterior chamber 92.

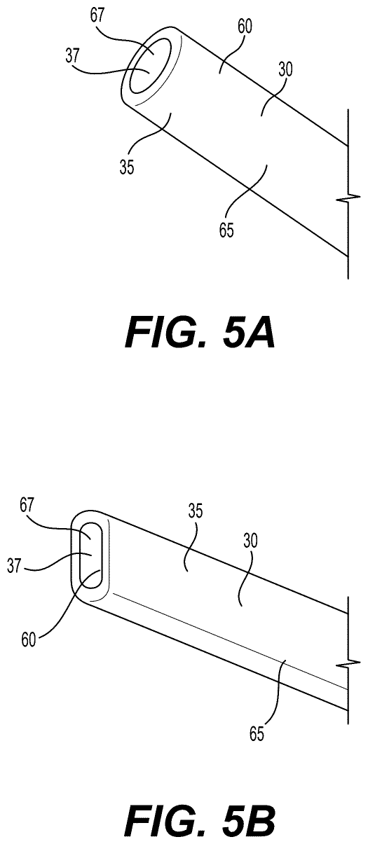

The cannula 30 includes a body 31, the proximal end 32, a distal end 33, an outer surface 35, and a lumen 37. The proximal end 32 of the cannula 30 is connected to the distal end 23 of the connector 20. The cannula 30 is between 10 mm and 40 mm in length, between 15 mm and 35 mm in length, between 10 mm and 30 mm in length, between 20 mm and 40 mm in length, between 20 mm and 35 mm in length, or between 30 mm and 40 mm in length. The gauge of the cannula can be between 16 and 34 G, 20 and 32 G, 20 and 30 G, or 20 and 26 G according to the Birmingham gauge. The outer diameter of the cannula is between 1.651 mm and 0.184 mm, 0.908 mm and 0.235 mm 0.908 mm and 0.311 mm, and 0.908 mm and 0.473 mm. The inner diameter of the lumen within the cannula is between 1.194 mm and 0.0826 mm, 0.603 mm and 0.108 mm, 0.603 mm and 0.159 mm, 0.603 mm and 0.127 mm. The wall thickness of the cannula 30 is between 0.229 mm and 0.0508 mm, 0.1524 mm and 0.0635 mm, 0.0508 mm and 0.0762 mm, 0.1524 mm and 0.1016 mm. As illustrated in FIGS. 5A and 5B, the cannula 30 may be round, oval shaped, or somewhat flat, while still maintaining an open lumen 37.

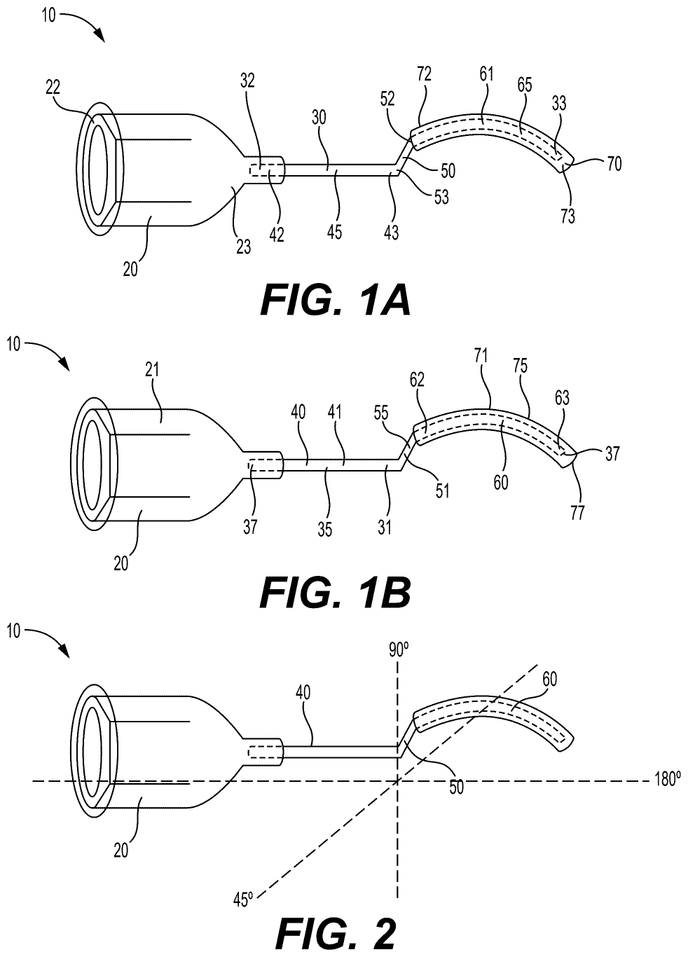

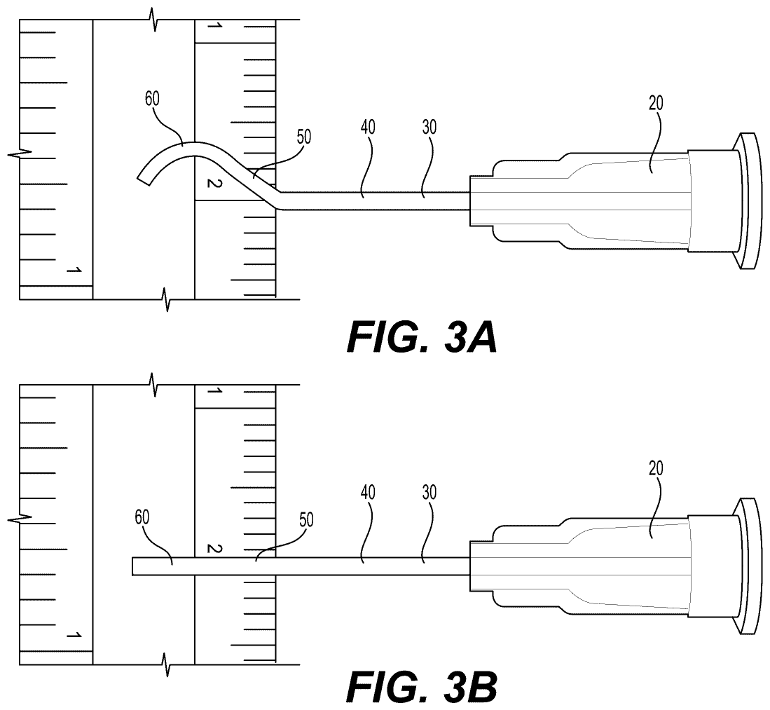

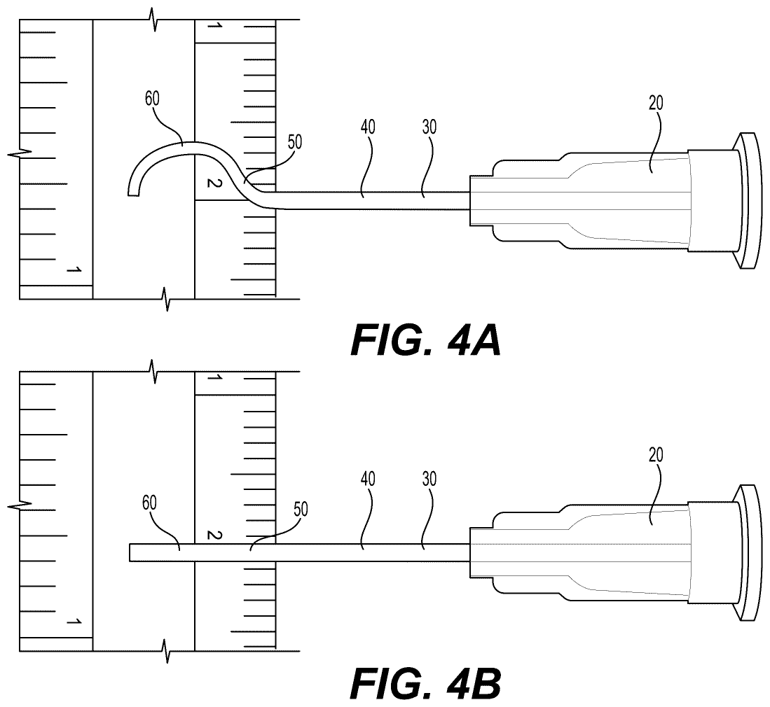

A cannula 30 is generally one continuous body. A cannula 30 can be made from one solid piece of material, or from a hollow material, depending on the desired characteristics of the device. Looking now at FIGS. 1-4 which illustrate various embodiments of the specific sections of the cannula, the proximal section 40 includes a body 41, a proximal end 42, a distal end 43, an outer surface 45, and a lumen (not illustrated). The proximal end 42 of the proximal section 40 is secured to the distal end 23 of the connector 20. The proximal section extends from the connector and is 5 mm to 35 mm, 5 mm to 30 mm, 5 mm to 25 mm, 10 mm to 30 mm, or 10 mm to 35 mm in length. The medial section 50 includes a body 51, a proximal end 52, a distal end of 53, an outer surface 55, and a lumen (not illustrated). The proximal end 52 of the medial section 50 is connected to the distal end 43 of the proximal section 40. The medial section 50 extends from the proximal section 40 at an angle of 90-170, 90, 100, 110, 120, 130, 140, 150, 160, or 170 degrees relative to the proximal section (See FIG. 2 for angle reference). The medial section 50 can be straight (FIGS. 1, 2, 3, and 6) or curved (FIGS. 4, 7, 8, and 9). When curved, the medial section 50 can be similar to and/or match the curvature of the distal section 60 (described below). The medial section is approximately 1 mm to 6 mm, 1 mm, 2 mm, 3 mm, 4 mm, 5 mm, or 6 mm in length. The distal section 60 includes a body 61, proximal end 62, a distal end 63, an outer surface 65, and a lumen 67. The proximal end 62 of the distal section 60 is connected to the distal end 53 of the medial section 50. The distal section 60 has an arcuate shape following the curvature of the inner cornea. The distal section 60 has a radius of curvature ranging from 4.0 mm to 10.0 mm, 4 mm, 5 mm, 6 mm, 6.4 mm, 6.5 mm, 6.6 mm, 6.7 mm, 7 mm, 7.5 mm, 8 mm, 9 mm, or 10 mm. The distal section has an arc length of 1 mm to 12 mm, 1 mm, 2 mm, 2.5 mm, 3 mm, 3.5 mm, 4 mm, 4.5 mm, 5 mm, 5.5 mm, 6 mm, 7 mm, 8 mm, 9 mm, 10 mm, 11 mm, or 12 mm.

The cannula 30 can be made of any material known in the art including, but not limited to, metal (stainless steel, tungsten carbide, titaniam, niobium, etc.), ceramic, polyetheretherketone (PEEK), or a combination thereof. The outer surface 35 of the cannula 30 may be either smooth or textured. The texturing may be on the proximal 40, medial 50 and/or the distal 60 sections of the cannula 30. Texture may be added to any and all parts of the cannula in any way known in the art including, but not limited to, laser etching, chemical etching, grinding, sand blasting, sanding, and machining. Texture can be added before, during and/or after the device 10 is manufactured. Various forms of surface texturing structures may be used including, but not limited to, dimples, grooves, dots, pits, pillars, and pyramids or a combination thereof. The surface textures can be microscopic textures. A variety of texture gradients may be utilized on the surface. The grit can range between 40 (400 μm) and 1200 (3.8 μm) according to the ANSI grading standard, between 50 and 1100 grit, between 100 and 1000 grit, between 200 and 1000 grit, between 300 and 1200 grit, between 500 and 1000 grit, or between 400 and 1100 grit. The distal section 60 is configured to remove endothelium and inner corneal lesions within an eye.

In certain embodiments of the present invention, a sleeve 70 is secured over one or more of the proximal 40, medial 50 and/or the distal 60 sections of the cannula 30 (See FIGS. 1 and 2). The sleeve 70 includes a body 71, a proximal end 72, distal end 73, an outer surface 75, and a lumen 77. The cannula 30 is inserted into the lumen 77 of the sleeve 70. The outer diameter of the sleeve 70 can be between 2.0 mm and 0.35 mm, 1.95 mm and 0.48 mm, 1.65 mm and 0.64 mm. The inner diameter of the sleeve 70 can be between 1.7 mm and 0.05 mm, 1.65 mm and 0.18 mm, 0.76 mm and 0.31 mm. The wall thickness of the sleeve 70 can be between 0.5 mm and 0.1 mm, 0.4 mm and 0.1 mm, 0.46 mm and 0.15 mm.

The sleeve 70 may be constructed of any material known in the art including, but not limited to, silicone, polyurethane, ceramic, poly-vinyl chloride (PVC), thermoplastic elastomer (TPE) fluoropolymers, fluorinated ethylene propylene (FEP), polyethylene, polyether ether ketone (PEEK), or a combination thereof. The outer surface 75 of the sleeve 70 may be either smooth or textured. The texturing can be achieved by any means known in the art as described previously. Various forms of surface texturing structures, texture gradients and grit size may be utilized as previously described. Texture of the sleeve may be added before, during or after the device 10 is manufactured. Texture may be added to the exterior of the cannula 30 through incorporation into a sleeve 70 which is fitted onto the cannula. In some embodiments, the sleeve 70 may extend beyond the cannula 20 for a range between 0.25 mm and 1.0 mm. In some embodiments, the sleeve 70 is tapered at its distal end 73.

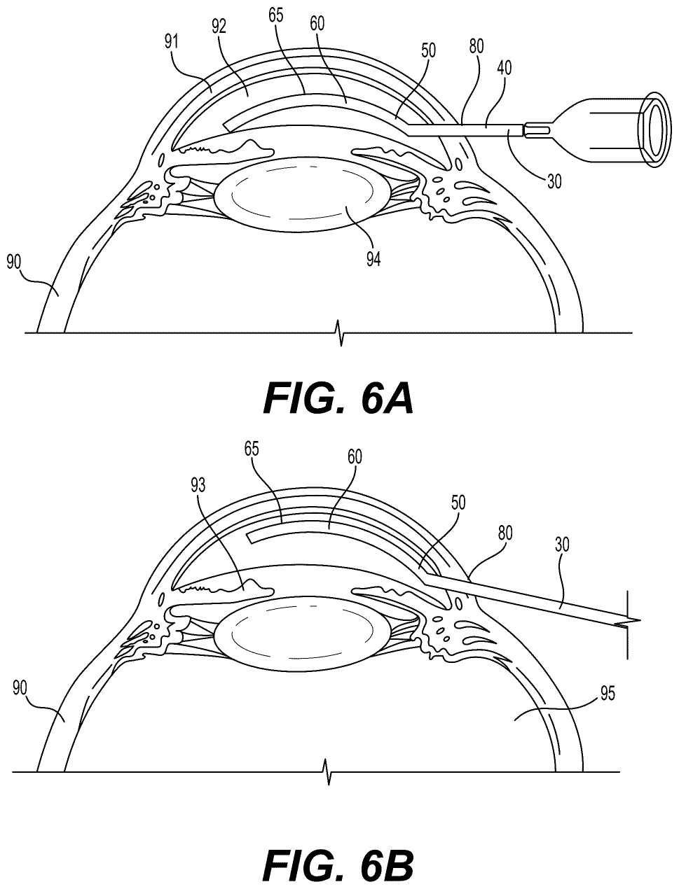

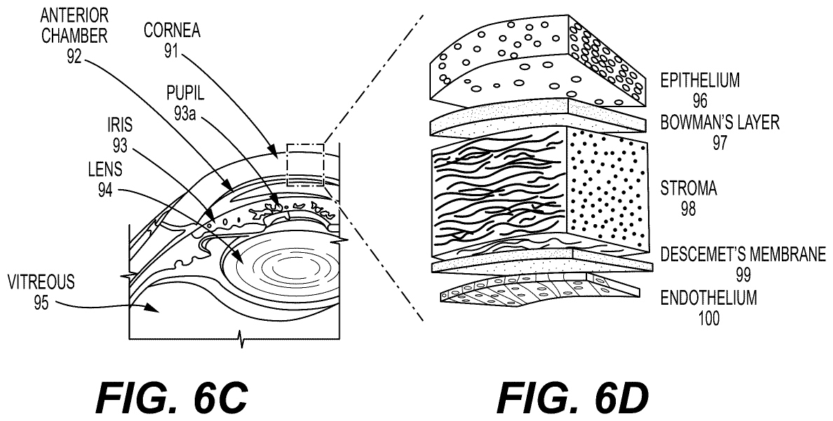

FIGS. 6, 7, and 9 illustrate the various layers of an eye 90 and the proper use of the medical device assembly 10 within the eye 90. As shown in the figures and as is known in the art, the human eye 90 includes a cornea 91, an anterior chamber 92, an iris 93, a pupil 93a, a lens 94, and vitreous fluid 95. Looking more closely at the cornea 91, we find layers that include the epithelium 96, Bowman's layer 97, the stroma 98, Descemet's membrane 99, and the endothelium 100. The distal end 60 of the medical device assembly 10 is inserted into the anterior chamber 92 between the iris 93 and the cornea 91. There, the medical device assembly 10 is used for removing corneal endothelium and inner corneal lesions while preserving Descemet's membrane.

The present invention also includes a method for removing one or more layers of the inner cornea which may include the endothelium, Descemet's membrane, Dua's layer, and the stroma. The removal of these layers may be complete or partial. The removal of these layers may also include the removal of lesions found in these layers including by not limited to guttae, cysts, vesicles, scars, mineral deposits, pigment deposits, cellular deposits, metabolic byproducts, dysfunction cells, debris, and other lesions found on the inner cornea. In some settings it would be appropriate to remove only the corneal endothelium and inner corneal lesions while preserving Descemet's membrane.

The layers of the cornea and associated corneal lesions are removed by engaging the inner cornea with the distal segment 60 of the device 10 in combination with movements. These movements can be a combination of one or more of rotational, horizontal, vertical and oblique movements. In some embodiments the movements of the distal segment along the inner cornea may cause the distal segment 60 to act as a polisher or wiper when moving along the inner layer to remove endothelium and lesions of the inner cornea. In some embodiments the movements of the distal segment along the inner cornea may cause the distal segment 60 to act as a rasp or screeder when moving along the inner layer to remove endothelium and lesions of the inner cornea. In some embodiments the mechanism of the removal of the inner corneal layers may be a combination of one or more of polishing, wiping, scraping, plowing, dislodging, rasping, among other methods.

The present invention includes a method for removing one or more layers of the inner cornea and associated corneal lesions, comprising:
a) supplying a medical device assembly 10;
b) inserting the cannula 30 into an anterior chamber 92;
c) moving the cannula 30 within the anterior chamber 92 while engaging the inner cornea;
d) removing corneal endothelium 100 and inner corneal lesions while preserving Descemet's membrane 99; and
e) withdrawing the cannula 30 from the anterior chamber 92.

In one embodiment of the above method, the connector 20 is supplied with constant or intermittent irrigation to maintain the depth of the anterior chamber 92. In another embodiment of the above method, the connector 20 is capped to prevent egress of fluid through the cannula 30 from the anterior chamber 92.

The above method may further comprise the step of:
adjusting a depth of insertion of the cannula 30 into the anterior chamber 92 to control an area of corneal endothelium and inner corneal lesions removal.

The above methods may further comprise the step of:
adjusting the angle of use of the cannula to raise and lower the distal section 60 to engage the cornea depending on the height of the anterior chamber to facilitate corneal endothelium and inner corneal lesions removal.

The above methods may further comprise the step of:
rotating the device 10 along the axis of the proximal section 40 to facilitate corneal endothelium and inner corneal lesions removal.

The above methods may further comprise the step of:
horizontal, oblique and vertical movements of the device 10, with the insertion site 80 of the cornea 91 acting as a pivot, to facilitate corneal endothelium and inner corneal lesions removal.

In some embodiments, the rotation of the device 10 along the axis of the proximal section 40 may be controlled to facilitate corneal endothelium and inner corneal lesions removal by the distal section 60. In some embodiments, the horizontal, oblique and vertical movements of the device 10 with the insertion site 80 as the pivot may be controlled to facilitate corneal endothelium and inner corneal lesions removal by the distal section 60. In some embodiments, the depth of insertion of the cannula 30 into the anterior chamber 92 may control the area of corneal endothelium and inner corneal lesions removal by the distal section 60.

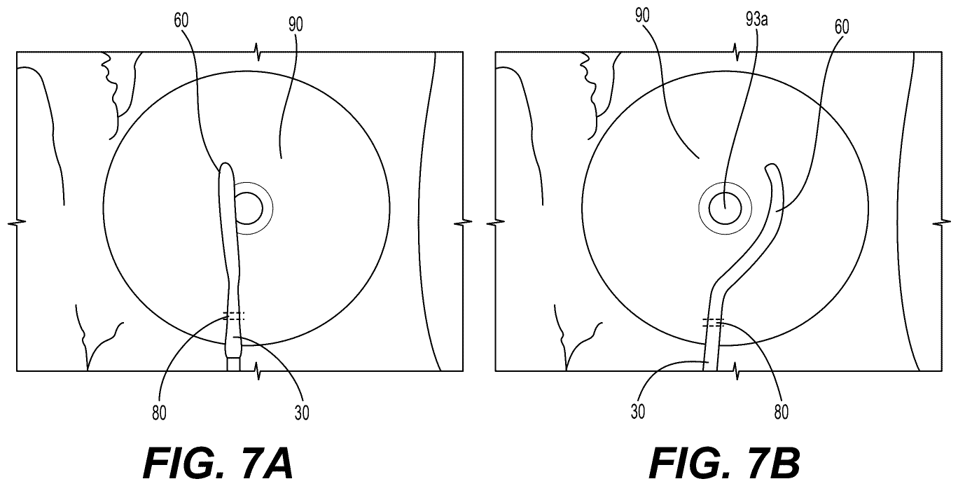

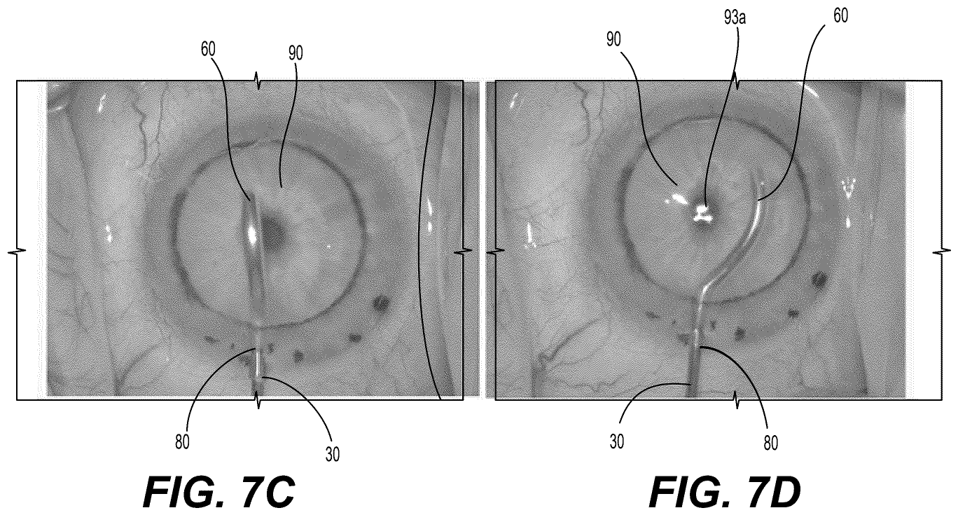

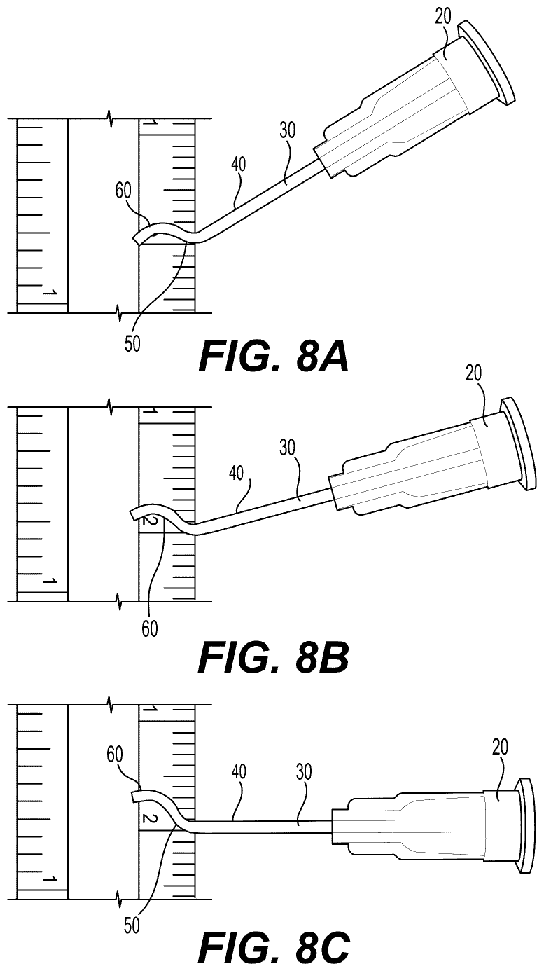

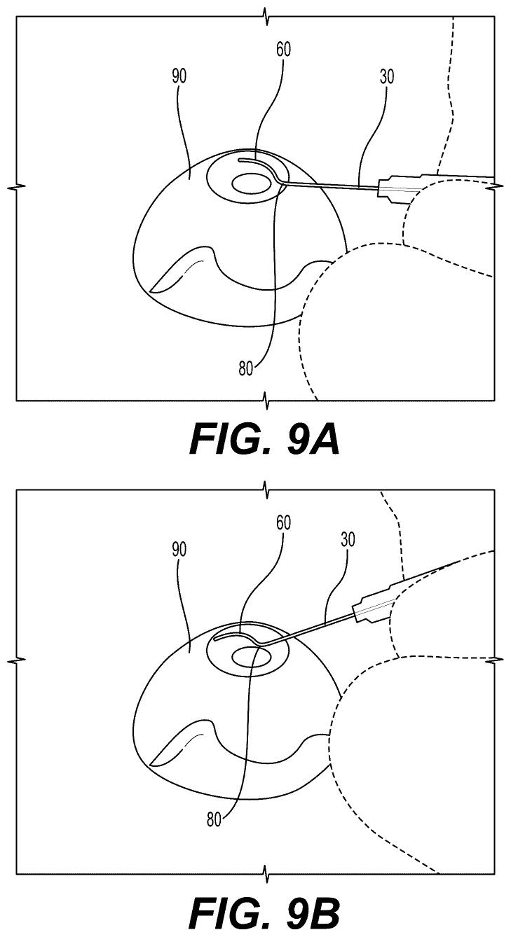

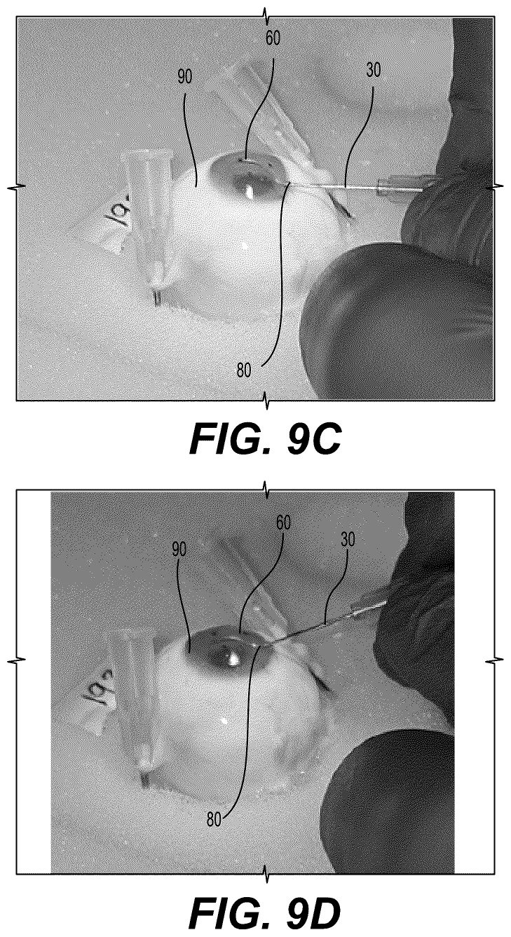

FIGS. 6-9 illustrate a method for removing corneal endothelium inner corneal lesions, according to some embodiments of the present invention. The method may include inserting a cannula 30 into the anterior chamber 92 of an eye 90 through a insertion site 80. The method may include rotating the proximal section 40 and connector 20 to initiate gentle wiping motions of the distal section 60 to remove corneal endothelium and inner corneal lesions. The wiping motion is illustrated in FIG. 7. FIG. 7C is the photo on which FIG. 7A is based. FIG. 7D is the photo on which FIG. 7B is based. FIGS. 8 and 9 illustrate how adjusting the angle of use of the device 10 adjusts the level within the anterior chamber 92 that the distal section 60 of the cannula 30 reaches. With the insertion site 80 of the cannula into an eye being equivalent in all three illustrations:
FIGS. 8A, 9B, and 9D illustrate a more shallow extension of the medial 50 and distal 60 sections by maintaining the proximal section 40 at a steeper angle for a shallow anterior chamber. FIG. 9D is the photo on which 9B is based.
FIG. 8B illustrates a less shallow extension of the medial 50 and distal 60 sections by maintaining the proximal section 40 at a less steep angle for a deeper anterior chamber. (See also FIGS. 9B and 9D).
FIGS. 8C, 9A, and 9C illustrate an even less shallow extension of the medial 50 and distal 60 sections by maintaining the proximal section 40 at little to no angle, allowing it to reach the highest level within the interior chamber. (See also FIGS. 9A and 9C). FIG. 9C is the photo on which 9A is based.

It is important to note that the above descriptions of exemplary embodiments of a medical device assembly 10 for removing cornel endothelium and inner corneal lesions are provided for illustrative purposes and should not be interpreted as limiting the scope of the medical device assembly for endothelium and inner corneal lesion removal.

Any method described herein may incorporate any design element contained within this application and any other document/application incorporated by reference herein.

To the accomplishment of the foregoing and related ends, one or more various embodiments include the features hereinafter fully described and particularly pointed out in the claims. The following description and the annexed drawings set forth in detail certain illustrative aspects and are indicative of but a few of the various ways in which the principles of the various embodiments may be employed. Other advantages and novel features will become apparent from the following detailed description when considered in conjunction with the drawings and the disclosed various embodiments are intended to include all such aspects and their equivalents.

Other aspects, advantages, and salient features of the disclosure will become apparent to those skilled in the art from the following detailed description, which, taken in conjunction with the annexed drawings, discloses various embodiments of the present disclosure.

While the present disclosure has been shown, and described with reference to various embodiments thereof, it will be understood by those ordinary skilled in the art that various changes in form and details may be made therein without departing from the scope of the present disclosure as defined by the appended claims and their equivalents.

What is claimed:

1. A medical device assembly comprising:
a connector including a body, a proximal end, and a distal end;
a cannula including a proximal section, a medial section, and a distal section;
wherein the proximal section is secured to the connector body;
wherein the medial section extends at an angle from the proximal section;
wherein the distal section extends from the medial section and has an arcuate shape following the curvature of the inner cornea; and
wherein the canula has at least one internal lumen.

2. The medical device of claim 1 wherein the connector is a handle, a lure lock hub, or a combination of both.

3. The medical device of claim 1 wherein a total length of the cannula is between 10 mm and 40 mm.

4. The medical device of claim 1 wherein a gauge of the cannula is between 20 and 32 G.

5. The medical device of claim 1 wherein an outer diameter of the cannula is between 0.908 mm and 0.235 mm.

6. The medical device of claim 1 wherein an inner diameter of the lumen within the cannula is between 0.603 mm and 0.108 mm.

7. The medical device of claim 1 wherein the wall thickness of the cannula is between 0.152 mm and 0.064 mm.

8. The medical device of claim 1 wherein the proximal section extends from the connector and is 5 mm to 30 mm in length.

9. The medical device of claim 1 wherein the medial section is oriented 90-170 degrees from the proximal section and is approximately 1 mm to 4 mm in length.

10. The medical device of claim 1 wherein the distal section has a radius of curvature ranging from 4.0 mm to 10.0 mm and has an arc length of 1 mm to 12 mm.

11. The medical device of claim 1 further comprising a sleeve secured over one or more of the proximal, medial and the distal sections of the cannula.

12. The medical device of claim 11 wherein an outer surface of the sleeve is textured.

13. The medical device of claim 1 wherein the material of the cannula is stainless steel.

14. The medical device of claim 1 wherein an outer surface of the cannula is textured.

15. The medical device of claim 1 wherein the connector is configured to connect to constant irrigation to maintain a depth of the anterior chamber.

16. The medical device of claim 1 wherein the distal section is configured to remove endothelium and inner corneal lesions within an eye.

17. A method for removing corneal endothelium and inner corneal lesions while preserving Descemet's membrane, comprising:
supplying the medical device assembly from claim 1;
inserting the cannula into an anterior chamber;
moving the cannula within the anterior chamber while engaging the inner cornea;
removing one or more inner corneal layers and inner corneal lesions; and
withdrawing the cannula from the anterior chamber.

18. The method of claim 17, further comprising the step of:
supplying the connector with constant irrigation to maintain the depth of the anterior chamber.

19. The method of claim 17, further comprising the step of:
adjusting a depth of insertion of the cannula into the anterior chamber to control an area of corneal endothelium and inner corneal lesions removal.

20. The method of claim 17, further comprising the step of:
adjusting the angle of use of the cannula to raise and lower the distal section to engage the cornea depending on the height of the anterior chamber in order to remove corneal endothelium and inner corneal lesions.

21. The method of claim 17, further comprising the step of:
rotating the proximal section while engaging the cornea to remove corneal endothelium and inner corneal lesions.
